(19) United States
(12) Patent Application Publication
(10) Pub. No.: US 2023/0157905 A1
(43) Pub. Date: May 25, 2023

(54) ELASTICIZED NONWOVEN LAMINATES

(71) Applicant: THE LYCRA COMPANY LLC, Wilmington, DE (US)

(72) Inventors: Hong LIU, Waynesboro, VA (US); Oguzhan CELEBI, Waynesboro, VA (US); Nicholas E. KURLAND, Staunton, VA (US)

(21) Appl. No.: 17/912,134

(22) PCT Filed: Mar. 24, 2021
(86) PCT No.: PCT/US2021/023798

§ 371 Date: Sep. 16, 2022

Related U.S. Application Data
(60) Provisional application No. 62/994,612, filed on Mar. 25, 2020.

(51) Int. Cl. A61F 13/49; D01F 6/70; A61L 15/26; C08G 18/10; C08G 18/76; C08G 18/48; C08G 18/32

(52) U.S. Cl. CPC A61F 13/4902; D01F 6/70; A61L 15/26; C08G 18/10; C08G 18/7671; C08G 18/4854; C08G 18/3228; D10B 2401/061; D10B 2401/063; D10B 2509/02; A61F 2013/49022

(57) ABSTRACT

Elasticized nonwoven laminates including high recovery power polyurethane elastic fiber, articles of manufacture with these elasticized nonwoven laminates and methods for production of the elasticized laminates and articles of manufacture are provided.

### FIELD

This disclosure relates to elasticized nonwoven laminates comprising a high recovery power polyurethane elastic fiber, articles of manufacture comprising these elasticized nonwoven laminates and methods for production of the elasticized laminates and articles of manufacture.

### BACKGROUND

The use of elastomeric fibers, filaments and/or films in, for example, leg bands and other components of disposable diapers has been known for many years. In a typical process to produce these components, spandex fibers or filaments or natural or synthetic rubber film strips are elongated to a specific draft and adhesively attached to, for example, one or two layers of a nonwoven substrate using a hot melt adhesive. This provides good stretch and recovery properties to the diaper component, e.g., the nonwoven substrate, which has been elasticized by incorporation of the elastomeric fibers, filaments and/or film into or onto this component.

The adhesives used in this process are frequently those which must be heated to an elevated temperature to form a good bond between the elastomeric fiber, filament or film and the material of the diaper component being elasticized, for instance a nonwoven fabric or substrate. At this elevated temperature, the break tenacity of the elastomeric fiber, filament or film is significantly lower than its break tenacity at room temperature (˜75° F.). If the break tenacity of the elastomeric fiber, filament or film at the elevated temperature experienced at the point of contact with the hot melt adhesive is lower than the first load force of the fiber, filament or film at room temperature and at the draft used in the elasticizing process, then the fiber, filament or film will break. It is thus commonly known that if the elastomeric fiber, filament or film is stretched to an excessive extent or if the adhesive is heated to too high a temperature when it contacts the elastomer, instances of breaks in the elastomeric fibers, filaments or film will occur during the process of preparing elasticized material for hygiene product components.

Typical process conditions for elasticizing material for diaper components with spandex and a hot melt adhesive involve use of a spandex fiber at a draft between 3.0 and 4.0 (200% to 300% elongation) and a standard elastic attachment hot melt adhesive temperature of about 260° F. to 325° F. (127° C. to 177° C.) when the adhesive is applied by a spiral spray or strand coating process. If the spandex draft is increased beyond 4.0 when the hot adhesive is applied, instances of breaks in the spandex at the point of adhesive application rapidly increase to an unacceptable level. If the adhesive temperature is decreased below about 260° F. (127° C.) to lessen the thermal load on the spandex fiber, the integrity of the bond between the spandex and the diaper component, e.g., nonwoven, decreases to an unacceptable level.

Breaks in the elastomeric fibers, filaments or film in the production of elasticized structures used for components of disposable hygiene products are highly undesirable. This is because when the elastomer breaks, the disposable product production line must be shut down; the elasticizing fibers, filaments or film must be re-strung; and the apparatus restarted. This causes significant down time, for example, of a diaper production line and generates a number of waste diapers.

U.S. Pat. No. 9,084,836 discloses articles of apparel or disposable hygiene products such as disposable diapers which include at least one relatively inelastic substrate, a polyurethane material selected form the group consisting of a film and one or more filaments including as the soft segment base of said polyurethane material a glycol which has a poly(tetramethylene-co-alkylene ether) structure comprising constituent units derived by copolymerizing tetrahydrofuran and a C₂ or C₃ alkylene oxide, wherein the portion of the units derived from C₂ or C₃ alkylene oxide comprises at least 15 mole % of said poly(tetramethylene-co-alkylene ether)glycol; and a hot melt adhesive having a temperature of from about 260° F. to about 350° F.

Notwithstanding the availability of components for disposable hygiene products which have been elasticized by the adhesive attachment or non-adhesive entrapment of spandex, it would be advantageous to identify additional stronger elastic materials for use in these products which are less prone to breakage. Specific advantages of such a material may include thermal stability, decreased consumption of elastic materials, greater efficiency in equipment operation (i.e., runtime per package), and improved sustainability in terms of emissions and transportation costs.

### SUMMARY

An aspect of the present invention relates to an elasticized nonwoven laminate which comprises a high recovery power polyurethane elastic fiber and a nonwoven laminate. In one nonlimiting embodiment, the high recovery power polyurethane elastic fiber is made of a polyol, an organic diisocyanate compound, and a diamine compound adhered to a nonwoven laminate. In one nonlimiting embodiment, the polyol has a minimum number average molecular weight of 450 and a maximum of 1800. In one nonlimiting embodiment, the polyol has a minimum number average molecular weight of 450 and a maximum of 1600.

Another aspect of the present invention relates to an article of manufacture, at least a portion of which comprises an elasticized nonwoven laminate with a high recovery power polyurethane elastic fiber within or juxtaposed with the nonwoven laminate. In one nonlimiting embodiment, the high recovery power polyurethane elastic fiber is made of a polyol, an organic diisocyanate compound, and a diamine compound. In one nonlimiting embodiment, the polyol has a minimum number average molecular weight of 450 and a maximum of 1800. In one nonlimiting embodiment, the polyol has a minimum number average molecular weight of 450 and a maximum of 1600.

Another aspect of the present invention relates to a method for producing an elasticized nonwoven laminate which comprises a high recovery power polyurethane elastic fiber within or juxtaposed with the nonwoven laminate. In one nonlimiting embodiment, the high recovery power polyurethane elastic fiber is made of a polyol, an organic diisocyanate compound, and a diamine compound. In one nonlimiting embodiment, the polyol has a minimum number average molecular weight of 450 and a maximum of 1800. In one nonlimiting embodiment, the polyol has a minimum number average molecular weight of 450 and a maximum of 1600.

### BRIEF DESCRIPTION OF FIGURES

FIG. 1 provides a comparison of yarn mechanical properties used in the present invention, relative to commercial examples of LYCRA HyFit® fiber.

FIG. 2. provides a comparison of 4-end laminate 0-150% third cycle mechanical characteristics with 3.8×draft relative to comparative commercial examples.

FIG. 3. provides a comparison of pre-stretched laminate retractive forces relative to commercial examples.

FIG. 4 provides a comparison of yarn mechanical properties of the obtained yarn of Examples 18 through 25.

### DETAILED DESCRIPTION

Provided by this disclosure are elasticized nonwoven laminates, articles of manufacture at least a portion of which comprise the elasticized laminate and methods for producing these elasticized nonwoven laminates and articles of manufacture.

In this disclosure, the nonwoven laminates are elasticized via a high recovery power polyurethane elastic fiber encompassed within or juxtaposed with the nonwoven laminate.

By “high-recovery power” polyurethane elastic fiber, it is meant a polyurethane elastic fiber having a normalized recovery force/unit decitex at 200% of the 5^{th} unload cycle, which is equal to or greater than 0.023 centinewtons (cN)/decitex (dtex). Use of high-recovery power polyurethane elastic fiber allows an overall decitex reduction compared to incumbent spandex fibers for non-woven laminate applications. In one nonlimiting embodiment, the decitex range is about 30 to 1500. In one nonlimiting embodiment, the decitex range is about 33 to 1100.

In one nonlimiting embodiment, the high recovery power polyurethane elastic fiber is made of a polyol, an organic diisocyanate compound, and a diamine compound to the nonwoven laminate.

Polyols with two or more different repeat units may be used by blending or copolymerizing. From the perspective of strength and recoverability, use of a polyol that blends poly(tetramethylene ether) glycol (PTMEG) and poly(tetramethylene-co-2-methyltetramethylene ether) glycol (3MCPG) is preferred. Other polyols may also be blended or copolymerized in any way as long as the properties of PTMEG, 3MCPG, or a polyol that blends these two types is maintained. Commercially available examples of suitable polyols include Terathane® 1000 and Terathane® 650 (The LYCRA Company of Wilmington, Del.).

Examples of polyether polyols that can be used include those glycols with two or more hydroxy groups, from ring-opening polymerization and/or copolymerization of ethylene oxide, propylene oxide, trimethylene oxide, tetrahydrofuran, and 3-methyltetrahydrofuran, or from condensation polymerization of a polyhydric alcohol, such as a diol or diol mixtures, with less than 12 carbon atoms in each molecule, such as ethylene glycol, 1,3-propanediol, 1,4-butanediol, 1,5-pentanediol 1,6-hexanediol, neopentyl glycol, 3-methyl-1,5-pentanediol, 1,7-heptanediol, 1,8-octanediol, 1,9-nonanediol, 1,10-decanediol and 1,12-dodecanediol. A linear, bifunctional polyether polyol is preferred. The polyol should have a number average molecular weight of about 450 to 1800. In one nonlimiting embodiment, the polyol has a number average molecular weight of about 450 to 1600. In one nonlimiting embodiment, a poly(tetramethylene ether) glycol of number average molecular weight of about 650 to about 1400 is used. The desired number average molecular weight may be achieved with a blend or mixture of two or more glycols which may be outside the desired molecular weight range.

In one nonlimiting embodiment, the polyol is a polyether-based polyol. In one nonlimiting embodiment, a low molecular weight polyol is blended with a high molecular weight polyol. In one nonlimiting embodiment, the polyol has a minimum number average molecular weight of 450 and a maximum of 1800. In one nonlimiting embodiment, the polyol has a minimum number average molecular weight of 450 and a maximum of 1600.

Aromatic, alicyclic, and aliphatic diisocyanate compounds can be used as the diisocyanates in the high recovery power polyurethane elastic fiber. Examples of aromatic diisocyanate compounds include, for example, diphenyl methane diisocyanate (hereinafter abbreviated as MDI), tolylene diisocyanate, 1,4-diisocyanate benzene, xylylene diisocyanate, and 2,6-naphthalene diisocyanate and the like. Examples of alicyclic and aliphatic diisocyanates include, for example, methylene bis(cyclohexyl isocyanate) (hereinafter abbreviated as H12MDI), isophorone diisocyanate, methyl cyclohexane 2,4-diisocyanate, methyl cyclohexane 2,6-diisocyanate, cyclohexane 1,4-diisocyanate, hexahydroxylylene diisocyanate, hexahydrotolylene diisocyanate, octahydro 1,5-naphthalene diisocyanate and the like.

These diisocyanates can be used individually, or two or more types can be used in combination.

An aromatic diisocyanate compound is preferably used from among these diisocyanate compounds for its excellent strength and heat resistance for elastic fibers, and use of MDI is more preferred. One or more other types of aromatic diisocyanate compounds may be blended with MDI and used. MDI may be a blend of the 2,4′ and 4,4′-MDI isomer. One suitable MDI composition contains at least 90% 4,4′-MDI isomer and preferably higher, such as Isonate 125 MDR™ from Dow Chemical, Desmodur® 44M from Bayer, Lupranate® M from BASF and Wannate® 11021N from Wanhua.

In one nonlimiting embodiment, the reaction equivalent ratio (molar ratio) of the organic diisocyanate compound to the polyol is less than 2. In one nonlimiting embodiment, the reaction equivalent ratio (molar ratio or capping ratio) of the diisocyanate compound to the polyol is greater than 1 but less than 2.

Diamine compounds are chain extenders for the high recovery power polyurethane elastic fiber of this disclosure. High recoverability becomes achievable when using diamine compounds.

Nonlimiting examples of diamine compounds which can be used include low molecular weight diamine compounds such as ethylene-diamine, 1,2-propanediamine, 1,3-propanediamine, 2-methyl-1,5-pentanediamine, 1,5-pentanediamine, 1,2-diaminebutane, 1,3-diaminebutane, 1-amino-3,3,5-trimethyl-5-aminomethylcyclohexane, 2,2-dimethyl-1,3-diaminopropane, 1,3-diamino-2,2-dimethylbutane, 2,4-diamino-1-methyl cyclohexane, 1,3-pentanediamine, 1,3 cyclohexane diamine, 1,4 cyclohexane diamine, bis(4-amino phenyl)phosphine oxide, hexamethylenediamine, 1,3-cyclohexyldiamine, hydrogenated meta-phenylene diamine (HMPD), 2-methyl pentamethylenediamine, 1,7-heptanediamine, 1,8-octanediamine, 1,9-nonanediamine, 1,10-decanediamine, 1,12-dodecanediamine, isophorone diamine, xylylenediamines, bis(4-amino phenyl) phosphine oxide and the like. One or more of these may be mixed and used. A low molecular weight diol compound such as ethylene glycol may be used together to the extent the properties are not damaged.

A diamine compound with 2 to 5 carbons is preferred, and when considering elastic yarn having superior elongation and elastic recovery and so forth, the use of ethylene diamine or a diamine mixture containing at least 70 mole % of ethylenediamine is particularly preferred. In addition to these chain extenders, a triamine compound (such as diethylene triamine or the like) may be used as well to form a branched structure to the extent that the effect of the present invention is not lost.

In one nonlimiting embodiment, a diamine compound such as ethylenediamine or its mixture with at least one diamine selected from the group consisting of an aliphatic diamine and an alicyclic diamine, each having 2 to 13 carbon atoms, is used.

In one nonlimiting embodiment, the polyurethane polymer is chain extended with a diamine compound and has a terminal group concentration of 5 to 50 mEq/kg of the polymer solids.

To control the molecular weight of the obtained polyurethane polymer, a chain terminator can be used at the time of the chain extension reaction. The mole ratio of the chain extender in regard to the chain terminator when considering stabilizing the yarn properties after spinning is preferred to be between 10 and 20, and more preferably between 14 and 18.

Nonlimiting examples of chain terminators that can be used include mono-alcohol compounds such as n-butanol, and monoamine compounds such as dimethylamine, diethylamine, n-propylamine, iso-propylamine, n-butylamine, cyclohexylamine, and n-hexylamine or a mixture thereof. A monoamine compound is preferred, while diethylamine is more preferred. Chain terminators are normally used by blending with chain extenders.

In one nonlimiting embodiment, at least one monoamine, primary or secondary, selected from the group consisting of an aliphatic amine and an alicyclic amine, each having 2 to 12 carbon atoms, is used.

A nonlimiting example of a high recovery power polyurethane elastic fiber useful in the nonwoven laminates and articles of manufacture of this disclosure is that described in U.S. Pat. No. 9,567,694, the disclosure of which is incorporated herein by reference in its entirety, which is made of a polyol with a molecular weight between 450 and 1600, an organic diisocyanate compound, and a diamine compound.

When using the solution polymerization method, a poly(urethane urea) solution can be obtained by performing polymerization using polyols, organic diisocyanate compounds, and diamine compounds and the like as raw materials within an organic solvent, for example, DMAc, DMF, DMSO, NMP, or a solution that uses these as primary components. This reaction method is also not particularly restricted, and examples include, a one-shot method in which each raw material is introduced into the solution and dissolved then heated to a suitable temperature to cause a reaction, or a prepolymer method in which a prepolymer is formed in a nonsolvent system by first reacting the polyol and the organic diisocyanate compound and afterwards dissolving the prepolymer in a solvent and reacting with the diamine compound for chain extension to synthesize poly(urethane urea). The prepolymer method is preferred.

Moreover, mixing one or two types of catalysts, such as an amine series catalyst and an organic metal catalyst, is preferred when synthesizing the polyurethane.

Examples of amine catalysts include N,N-dimethylcyclohexylamine, N,N-dimethylbenzyl amine, triethyl amine, N-methylmorpholine, N-ethylmorpholine, N,N,N′,N′-tetramethylethylene diamine, N,N,N′,N′-tetramethyl-1,3-propanediamine, N,N,N′,N′-tetramethylhexane diamine, bis-2-dimethylamineethylether, N,N,N′,N′-pentamethyldiethylenetriamine, tetramethylguanidine, triethylenediamine, N,N′-dimethylpiperazine, N-methyl-N′-dimethylaminoethyl-piperazine, N-(2-dimethylaminoethyl)moropholine, 1-methylimidazole, 1,2-dimethylimidazole, N,N-dimethylaminoethanol, N,N,N′-trimethylaminoethylethanolamine, N-methyl-N′-(2-hydroxyethyl)piperazine, 2,4,6-tris(dimethylaminomethyl)phenol, N,N-dimethylaminihexanol, and triethanolamine, and the like.

In one nonlimiting embodiment, the high recovery power polyurethane elastic fiber is spun from a solution-polymerized polyurethane polymer solution by a prepolymer method.

In accordance with this disclosure, the high recovery power polyurethane elastic fiber is used to elasticize a nonwoven laminate. The high recovery power polyurethane elastic fiber may be encompassed within or juxtaposed with the nonwoven laminate.

In one nonlimiting embodiment, the high recovery power polyurethane elastic fiber is first elongated and then applied or incorporated within the nonwoven laminate in its elongated state.

This process of elasticizing a nonwoven typically incorporates from 2 to 50 ends or more into the nonwoven laminate. Once adhesively bonded within or juxtaposed with the nonwoven laminate, the elongated fiber is allowed to relax, to thereby provide the elastic nonwoven laminate. In one nonlimiting embodiment, the high recovery power polyurethane elastic fiber is elongated in at least one direction to a draft of from about 3.0 to about 4.0. The elasticized nonwoven laminate is suitable for use in a disposable hygiene product.

Nonwoven laminates which are elasticized in accordance with this disclosure can be any type of flexible structure that can be used as or converted into components which, in one embodiment, are useful for incorporation into or onto disposable hygiene products. Disposable personal hygiene products can be any product which serves to facilitate, improve, enhance or preserve the hygiene of persons or animals using the product. Non-limiting examples of disposable hygiene products include disposable diapers; training pants; adult incontinence devices and products; catamenial devices, garments and products; bandages; wound dressings; surgical drapes, surgical gowns, surgical or other hygienic protective masks, hygienic gloves, head coverings, head bands, ostomy bags, bed pads, bed sheets, and the like.

Such products may or may not be useful for also absorbing body fluids. Products of this type are further generally disposable in the sense that they are used only once or at most a few times and/or for only a relatively short period of time and are then discarded. They are generally not washed, cleaned, refurbished or reconditioned and then reused.

The components which are made from the nonwoven laminates elasticized in accordance with the process herein can be used as or in elements found within disposable hygiene products of the foregoing types. Such elements can include, for example, front, back and side panels, leg cuffs, leg holes, belly bands, and/or waist bands of diapers or training pants. These hygiene product components can be prepared, for example, by converting the elasticized nonwoven laminate as prepared herein in bulk form into separate individual segments of size and configuration suitable for incorporation into individual disposable personal hygiene products.

The elasticized nonwoven laminates prepared in accordance with the process herein will comprise at least one relatively inelastic nonwoven laminate. For purposes of this invention, the term “nonwoven laminate” is used interchangeably with the term “nonwoven substrate” and in their broadest sense are meant to include any flexible or deformable nonwoven substrate which has at least one surface onto which the high recovery power polyurethane elastic fiber can be adhesively bonded. Such nonwoven laminates will generally be flexible but relatively inelastic substrates with two surfaces, e.g., upper and lower. By “relatively inelastic substrate” it is meant that it can be elongated no more than about 120% in any direction without rupture or those which exhibit growth of more than 30% of the elongated length after elongation to 50% of the break elongation and removal of the elongating force.

Relatively inelastic substrates for elasticizing herein are in the form of nonwoven substrates. Nonwoven substrates or “webs” are substrates having a structure of individual fibers, filaments or threads that are interlaid, but not in an identifiable, repeating manner. Nonwoven substrates can be formed by a variety of conventional processes such as, for example, melt blowing processes, spunbonding processes and bonded carded web processes.

Melt blown substrates or webs are those made from melt blown fibers. Melt blown fibers are formed by extruding a molten thermoplastic material through a plurality of fine, usually circular, die capillaries as molten thermoplastic material or filaments into a high velocity gas (e.g. air) stream. This attenuates the filaments of molten thermoplastic material to reduce their diameter, which may be to microfiber diameter. Thereafter, the melt blown fibers are carried by the high velocity gas stream and are deposited on a collecting surface to form a web of randomly disbursed melt blown fibers. Such a process is disclosed, for example, U.S. Pat. No. 3,849,241, which patent is incorporated herein by reference.

Spunbonded substrates or “webs” are those made from spunbonded fibers. Spunbonded fibers are small diameter fibers formed by extruding a molten thermoplastic material as filaments from a plurality of fine, usually circular, capillaries of a spinneret. The diameters of the extruded filaments are then rapidly reduced as by, for example, eductive stretching or other well-known spun-bonding mechanisms. The production of spun-bonded nonwoven webs is illustrated, for example, in U.S. Pat. Nos. 3,692,618 and 4,340,563, both of which patents are incorporated herein by reference.

The relatively inelastic laminates to be elasticized by the process of this invention can be constructed from a wide variety of materials. Nonlimiting examples of suitable materials include polyethylene, polypropylene, polyesters such as polyethylene terephthalate, polybutane, ethylenepropylene co-polymers, polyamides, tetrablock polymers, styrenic block copolymers, polyhexamethylene adipamide, poly-(oc-caproamide), polyhexamethylenesebacamide, polyvinyls, polystyrene, polyurethanes, polytrifluorochloroethylene, ethylene vinyl acetate polymers, polyetheresters, cotton, rayon, hemp and nylon. In addition, combinations of such material types may be employed to form the relatively inelastic laminates to be elasticized herein.

Preferred nonwoven laminates to be elasticized herein include structures such as polymeric spunbonded nonwoven webs. Particularly preferred are spunbonded polyolefin nonwoven webs having a basis weight of from about 10 to about 40 grams/m². More preferably such structures are polypropylene spunbonded nonwoven webs having a basis weight of from about 10 to about 25 grams/m².

In one nonlimiting embodiment, the high recovery power polyurethane elastic fiber is adhesively bonded or attached to the relatively inelastic substrate being elasticized. Adhesive bonding of the high recovery power polyurethane elastic fiber to such inelastic flexible substrates in accordance with the process herein is generally brought about through the use of a conventional hot melt adhesive.

Conventional hot melt adhesives are typically thermoplastic polymers which exhibit high initial tack, provide good bond strength between the components and have good ultraviolet and thermal stability. Preferred hot melt adhesives will be pressure sensitive. Examples of suitable hot melt adhesives are those comprising a polymer selected from the group consisting of styrene-isoprene-styrene (SIS) copolymers; styrene-butadiene-styrene (SBS) copolymers; styrene-ethylene-butylene-styrene (SEBS) copolymers; ethylene-vinyl acetate (EVA) copolymers; amorphous poly-alpha-olefin (APAO) polymers and copolymers; and ethylene-styrene interpolymers (ESI). Most preferred are adhesives based on styrene-isoprene-styrene (SIS) block copolymers. Hot melt adhesives are commercially available. They are marketed under designations such as H-2104, H-2494, H-4232 and H-20043 from Bostik; HL-1486 and HL-1470 from H.B. Fuller Company.

In accordance with the process of the present invention, in this nonlimiting embodiment, the high recovery power polyurethane elastic fiber described herein will be elongated in at least one direction and while in the elongated condition will be adhesively bonded to at least one of the relatively inelastic nonwoven laminates which are to be elasticized. Generally, in this step of the process, the high recovery power polyurethane elastic fiber will be stretched to a draft of from greater than about 3.0×(200% elongation) to about 4.0×(300% elongation) prior to bonding with the relatively inelastic substrate.

Drafting of the high recovery power polyurethane elastic fiber to the desired extent can be brought about by the application of stretching force to the fiber in the machine direction. In commercial production operations, such elongating force can be applied by means of adjustment of the speed of, and/or tensioning force applied by, the feed rolls of the high recovery power polyurethane elastic fiber and the wind-up rolls for the elasticized product being produced. Sets of tensioning rollers may also be employed to provide or assist in polyurethane elongation.

Also provided, generally concurrently with provision of the elongated high recovery power polyurethane elastic fiber, will be at least one type of relatively inelastic nonwoven substrate as described herein, to which the elongated high recovery power polyurethane elastic fiber is to be adhesively bonded within or juxtaposed therewith. Like the high recovery power polyurethane elastic fiber, the inelastic substrate material can be provided from feed rolls.

Frequently, the high recovery power polyurethane elastic fiber is bonded within or juxtaposed with more than one substrate to form multilayer laminates. A preferred composite laminate structure of this type is described more fully hereinafter.

In one nonlimiting embodiment, after or as the high recovery power polyurethane elastic fiber has been or is being elongated, and before, as, or even after the high recovery power polyurethane elastic fiber is contacted with substrate(s) being elasticized, a hot melt adhesive is applied, e.g., sprayed or coated onto one or more of the surfaces of the high recovery power polyurethane elastic fiber and/or the substrate(s) of the structure being elasticized. The surfaces of the elongated high recovery power polyurethane elastic fiber and the relatively inelastic substrate(s) are then brought into and maintained in contact with each other in any suitable manner such that at least some adhesive material is interposed between at least some portions of the surfaces of the elements which are to be bonded together.

In one nonlimiting embodiment, the hot melt adhesive is applied to the surface of the high recovery power polyurethane elastic fiber and/or the substrate(s) being elasticized in a manner which forms a continuous coating of adhesive on such surfaces. In fact, the hot melt adhesive can be applied in a variety of different ways. In one method, the melted adhesive can be deposited as a discontinuous web from a spray nozzle, a process known as melt blowing. In another method, the melted adhesive can be deposited as a solid stream from a nozzle which moves in a spiral pattern as the materials to be bonded pass by the nozzle. Such a technique is known as spiral spray. Adhesive dispensed via a spray nozzle in melt-blowing or spiral spray processes can be propelled through the nozzle by means of jets of heated air which can be externally heated to temperatures at or above the melt temperature of the adhesive. Adhesive can also be applied to any of the desired surfaces in a “dot matrix” pattern or applied directly to the fiber and/or to the nonwoven laminate by direct coating or spray technology.

The temperature of the hot melt adhesive at its point of contact with the high recovery power polyurethane elastic fiber depends on the temperature of the adhesive as dispensed, the amount of adhesive used, the adhesive application technology, and the specific details of the physical arrangement of the system used to apply the adhesive. Normally, the temperature of the adhesive as it leaves the application head is used as the benchmark to define the adhesive temperature used in the process herein since the temperature of the adhesive at the time of its actual contact with the polyurethane fibers or film is hard to measure. However, it is understood that the temperature of the adhesive when it contacts the polyurethane can range from a value essentially equal to the adhesive temperature as it leaves the application head (such as in slot coat or other strand application systems such as the Sure Wrap® system made by Nordson, Inc.) to a value which is as much as 70° F. to 150° F. lower than the adhesive temperature as it leaves the application head, such as in the case of spiral spray or melt blown application systems.

The temperature of the adhesive as it leaves the application head in the process herein will generally be within the range of from about 280° F. to about 350° F. Preferably, the hot melt adhesive used is one which should be provided at a melt temperature of from about 300° F. to about 325° F. Contact of the hot melt adhesive which is within such temperature ranges at the time of contact with the polyurethane material can frequently bring the temperature of the polyurethane to a value within the range of from about 125° F. to about 300° F. At such temperatures, the selected polyurethane materials used herein, i.e., those which are based on poly(tetramethylene-co-alkylene ether)glycols, can be drafted to the extent specified herein without exhibiting an unacceptable incidence of breaks in the high recovery power polyurethane elastic fiber.

After the adhesive has been applied to the appropriate surfaces, the high recovery power polyurethane elastic fiber and the substrate(s) being elasticized are then maintained in contact with each other under conditions sufficient to adhesively bond the elongated high recovery power polyurethane elastic fiber within or juxtaposed with the relatively inelastic substrate(s). This is generally carried out by applying pressure to the contacted materials via the processing apparatus being used in order to form the adhesive bonding between the materials. For example, the contacted high recovery power polyurethane elastic fiber and nonwovens can be passed through a pair of nip rollers prior to being further processed and/or before being taken up on wind up rolls.

After the high recovery power polyurethane elastic fiber has been adhesively bonded in its elongated state within or juxtaposed with the relatively inelastic substrate(s), the resulting elasticized composite structure is allowed to relax by removing the tension which has kept the polyurethane material elongated. This allows the resulting elastic nonwoven laminate to retract, thereby forming a gathered or puckered composite structure which is stretchable, and which can be converted into elastic components for disposable hygiene products or articles of apparel.

In one particular preferred embodiment, a composite nonwoven laminate is prepared which comprises two outer layers of nonwoven substrates of substantially equal width and an inner layer of substantially parallel, equally spaced, high recovery power polyurethane elastic fiber. Both of the nonwoven substrates used in such preferred composite nonwoven laminate can be made of synthetic polymeric fibers such as polyolefin, polyester or polyamide fibers. Both of these nonwoven substrates will frequently be thermally bonded, spunbonded or hydroentangled webs. They can have basis weight values ranging from about 10 to about 30 grams/m². The three layers of such preferred composite laminate structures are bonded together by a hot melt adhesive composition which constitutes from about 5% to about 50% by weight of the composite laminate structure. Preparation of the composite structures described herein can be carried out using conventional apparatus and processing techniques. Such apparatus and techniques are disclosed, for example, in U.S. Pat. Nos. 4,634,482; 4,720,415; 4,482,666; 6,491,776; and 6,713,415; in U.S. Patent Publication No. 2002/0119722; and in PCT Publication No. WO 80/00676, all of which patent publications are incorporated herein by reference.

The elastic nonwoven laminates prepared in accordance with the process herein can be used as, or subsequently converted into, stretchable components for use in articles of manufacture such as disposable hygiene products. This conversion will typically involve cutting the elastic nonwoven laminates into lengths and configurations suitable for the particular type of hygiene product in which such components will be used. Such conversion procedures are conventional and can be carried out at the time and location of preparation of the composite structures herein. Alternatively, preparation of the composite structures herein, and/or conversion of such composite structures prepared elsewhere, into hygiene product can be carried out in connection with the production of the disposable hygiene articles into which the elasticized components are to be incorporated, e.g., at, near or as part of a diaper production line.

An alternative method to using hot melt elastic attachment adhesives previously described is to use ultrasonic bonding or other thermo-mechanical means to melt the nonwoven substrate juxtaposed with the elongated fiber or around the elongated fiber to entrap the fiber within the nonwoven substrate.

As will be understood by the skilled artisan upon reading this disclosure, other articles of manufacture comprising a nonwoven laminate, i.e. in addition to diapers, could be enhanced with this technology to create new consumer value.

The following example demonstrates the present disclosure and its capability for use in producing diapers which stay in place without inclusion or with decreased amounts of spandex or rubber fiber. The invention is capable of other and different embodiments, and its several details are capable of modification in various apparent respects, without departing from the scope and spirit of the present invention. Accordingly, the examples are to be regarded as illustrative and not as restrictive.

### EXAMPLES

Having described the embodiments of the present disclosure, in general, the following Examples describe some additional embodiments of the present disclosure. While embodiments of present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Material List

Terathane® PTMEG 1800 is a poly(tetramethylene ether) glycol, with a number average molecular weight of 1800 grams/mole, supplied by The LYCRA Company (Wilmington, Del., United States).

Terathane® PTMEG 1400 is a poly(tetramethylene ether) glycol, with a number average molecular weight of 1400 grams/mole, supplied by The LYCRA Company (Wilmington, Del., United States).

Terathane® PTMEG 1000 is a poly(tetramethylene ether) glycol, with a number average molecular weight of 1000 grams/mole, supplied by The LYCRA Company (Wilmington, Del., United States).

Terathane® PTMEG 650 is a poly(tetramethylene ether) glycol, with a number average molecular weight of 650 grams/mole, supplied by The LYCRA Company (Wilmington, Del., United States).

Isonate® 125MDR or MDI is a mixture of diphenylmethane diisocyanate containing 98% 4,4′-MDI isomer and 2% 2,4′-MDI isomer (commercially available from the Dow Company, Midland, Mich.).

EDA stands for ethylenediamine as a chain extender; DEA stands for N,N-diethylamine as the chain terminator; DMAc stands for N,N-dimethylacetamide as the solvent.

### Test Methods

The viscosity of the polymer solutions was determined in accordance with the method of ASTM D1343-69 with a Model DV-8 Falling Ball Viscometer (Duratech Corp., Waynesboro, Va.) operated at 40° C. and reported as poises.

The solid content in the polymer solutions was measured by a microwave heated moisture/solids analyzer, Smart System 5 (CEM Corp., Matthews, N.C.).

Percent isocyanate (% NCO) of the capped glycol prepolymer was determined according to the method of S. Siggia. “Quantitative Organic Analysis via Functional Group”, 3rd Edition, Wiley & Sons, New York, pages 559-561 (1963) using a potentiometric titration.

The strength and elastic properties of the spandex fibers were measured in accordance with the general method of ASTM D 2731-72. Three fibers, a 5.0 cm gauge length and a 0-300% elongation cycle were used for each of the measurements. The samples were cycled five times at a constant elongation rate of 50 centimeters per minute. Load power (1TP200), the stress on the spandex during the first cycle at 200% extension, is reported as centinewtons for a given decitex (cN/dtex). Unload power (5TM200) is the stress at an extension of 200% for the fifth unload cycle and is also reported in centinewtons force. Percent elongation at break was measured on a sixth extension cycle.

Normalized recovery is expressed as recovery on the 5^{th} unload cycle, at 200% elongation (5TM200), which has been normalized to the unit fineness of the fiber (i.e., decitex).

Percent set was also measured on samples that had been subjected to five 0-300% elongation/relaxation cycles. The percent set, % SET, was then calculated as

% SET=100×(L_{f}−Lₒ)/L₀

where Lₒ and L_{f} are respectively the fiber length when held straight without tension before and after the five elongation/relaxation cycles.

Elasticized nonwoven laminates are produce for testing in a 4-thread laminate form, via high-speed lamination to the nonwoven. The High-Speed Laminator is a device which produces nonwoven—spandex—nonwoven laminates using a process that simulates the process commonly used on high speed diaper production lines. This type of nonwoven—spandex fiber—nonwoven laminate is commonly made as part of the construction of a disposable diaper. In the process carried out with the High-Speed Laminator, spandex fibers are elongated to a specific draft (in this nonlimiting example 3.8×) or tension and guided in parallel, evenly spaced apart configuration to a position immediately above a sheet of a low basis weight nonwoven (commonly called the back sheet). A hot melt adhesive is applied by standard strand coating application technology hereinafter described. The spandex fibers are then brought into direct contact with the nonwoven sheet, and a second nonwoven sheet (commonly known as the top sheet) is brought into direct contact with bottom sheet/spandex fibers assembly. The components are thus layered in the order top sheet—spandex fibers—bottom sheet, and the entire assembly is passed through a nip roll. In this example, the adhesive application technology used in the laminator is a Sure Wrap® nozzle made by Nordson, Inc. of Dawsonville, Ga. In the setup using this type of apparatus, the tip of the nozzle is in contact with the spandex fibers, and the spandex fibers at point of adhesive application can be between 0.25 and 0.5 inch above the back sheet. The linear distance between the point of adhesive application and the nip roll is generally about 8 inches, and the linear speed of the machine can commonly be run between 200 and 1000 feet per minute. The various nonwoven, adhesive and spandex materials used, along with the specific test conditions employed in this example, are as follows: Nonwoven: 15 grams/m² spunbond polypropylene made by Avgol, Inc. Adhesive: H-4232 or H-20043 elastic attachment hot melt adhesive made by Bostick, Inc.

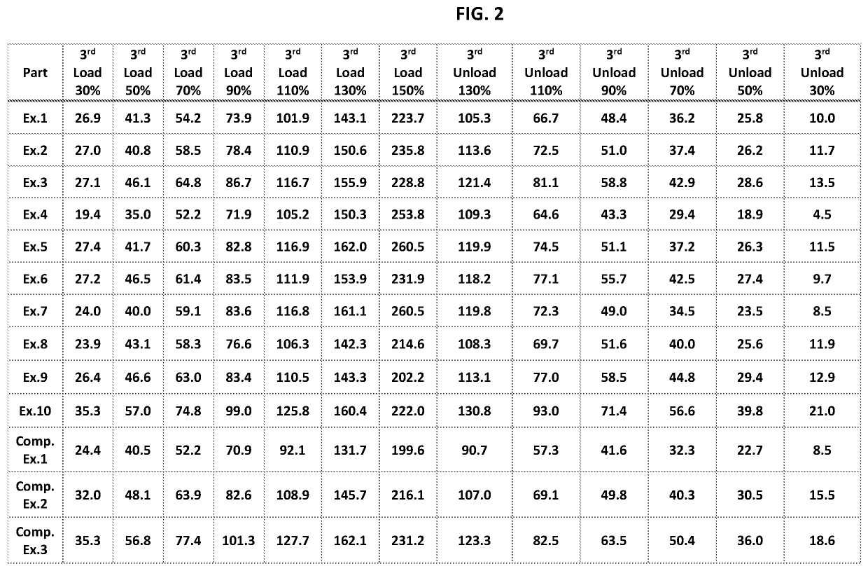

The strength and elastic properties of the elasticized nonwoven laminates were measured on the same tensile testing equipment use for spandex fiber analysis. A single laminate sample, a 7.6 cm gauge length, and a 0-150% elongation cycle was used for each of the measurements. The samples were cycled three times at a constant elongation rate. During this test, load and unload powers were recorded at 10% intervals on all three cycles, in units of centinewtons. An excerpt of this test data is reported in FIG. 2, which details select 3^{rd} cycle load and unload data.

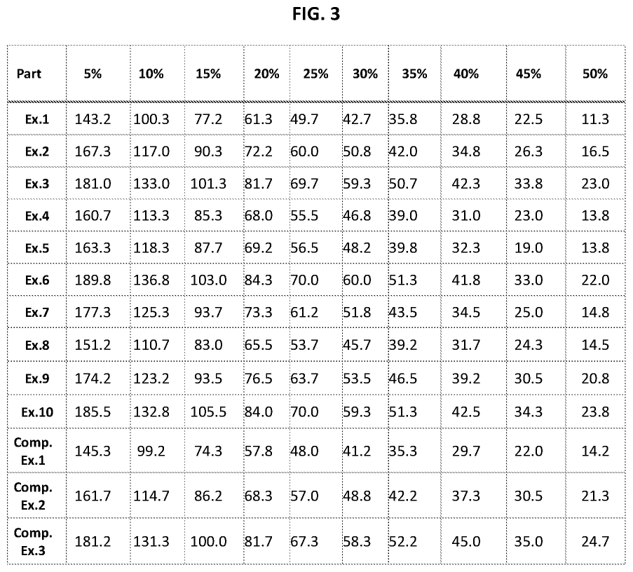

Laminate retractive force testing (as in FIG. 3) is a modification of the method used for laminate cycling, and utilizes standard tensile testing equipment. A 22-centimeter length of laminate, pre-tensioned to 392 centinewtons, is clamped into tensile-testing equipment equipped with a 20 centimeter gauge length. The laminate sample is cycled three times at a constant elongation rate, and cycles are conducted to 0 centinewtons load, then back to the initial gauge length. On the third cycle, laminate retractive force is recorded at 5% contraction intervals down to 50% contraction from the initial gauge length.

### Example 1

A prepolymer was obtained by reacting polytetramethylene ether glycol (PTMEG) with a molecular weight of 1411 g/mole with 4,4′-diphenylmethane diisocyanate (MDI) at a 3.62:1.00 (weight by weight), respectively, in a continuous polymerization reactor under neat conditions at 80° C. for 3 h at a specified reaction rate. The residual isocyanate group after the reaction was 2.77 weight %. The capping ratio (mole ratio of isocyanate to glycol) was 1.55. 124.40 grams of the obtained prepolymer were dissolved in 195.93 grams of DMAc at 60° C., and the chain extender solution in which 2.21 grams of ethylenediamine and 0.32 grams of diethylamine and 73.83 grams DMAc was added while stirring vigorously at 80° C. to obtain a viscosity adjusted polymer solution of 32 weight % concentration.

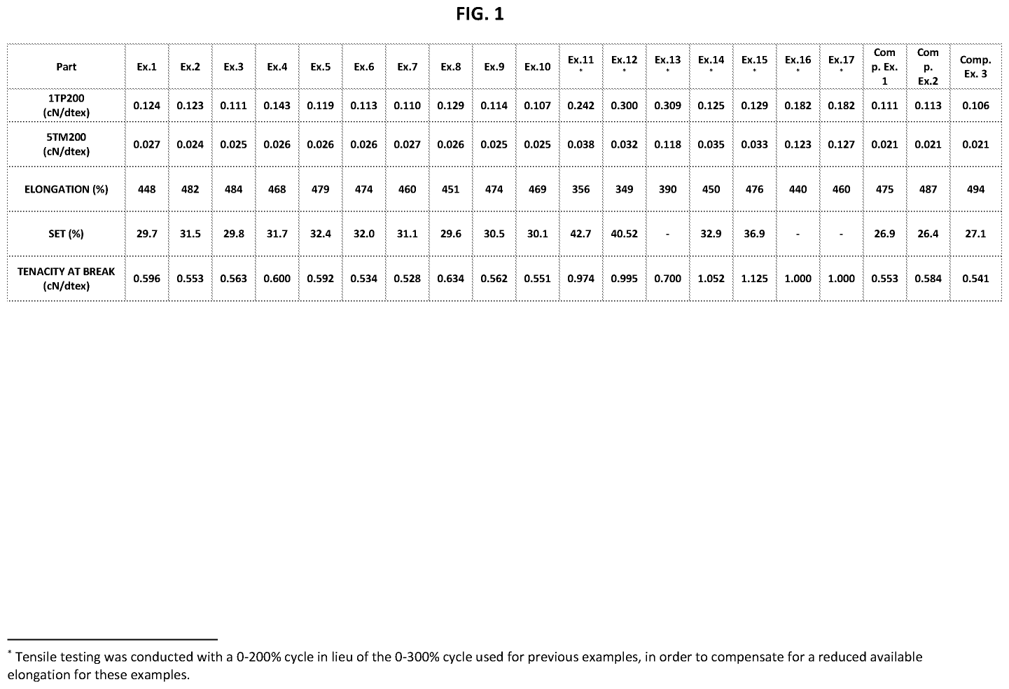

The polymer was blended at 94 parts by weight of the polyurethane polymer solids with 6 parts by weight of the additive solids to make the spinning concentrate solution. This was dry spun at a speed of 640 m/min with a speed ratio for the Godet roll to winding machine at 1.30 to obtain 530 decitex multiple filament yarn. The yarn mechanical properties of the obtained yarn are shown in FIG. 1. Properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 2

The same procedures and ingredients as Example 1 were used, but the produced fiber conditions were adjusted to a spinning speed of 619 m/min, with a speed ratio for the Godet roll to winding machine at 1.25 to obtain a 600 decitex multiple filament yarn. The yarn mechanical properties of the obtained yarn are shown in FIG. 1. Properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 3

The same procedures and ingredients as Example 1 were used, but the produced fiber conditions were adjusted to a spinning speed of 488 m/min, with a speed ratio for the Godet roll to winding machine at 1.25 to obtain a 670 decitex multiple filament yarn. The yarn mechanical properties of the obtained yarn are shown in FIG. 1. Properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 4

A prepolymer was obtained by reacting polytetramethylene ether glycol (PTMEG) with a molecular weight of 1432 g/mole with 4,4′-diphenylmethane diisocyanate (MDI) at a 3.55:1.00 (weight by weight), respectively, in a continuous polymerization reactor under neat conditions at 80° C. for 3 h at a specified reaction rate. The residual isocyanate group after the reaction was 3.16 weight %. The capping ratio (mole ratio of isocyanate to glycol) was 1.58. 130.62 grams of the obtained prepolymer were dissolved in 207.05 grams of DMAc at 60° C., and the chain extender solution in which 2.50 grams of ethylenediamine and 0.32 grams of diethylamine and 76.61 grams DMAc was added while stirring vigorously at 80° C. to obtain a viscosity adjusted polymer solution of 32 weight % concentration.

The polymer was blended at 94 parts by weight of the polyurethane polymer solids with 6 parts by weight of the additive solids to make the spinning concentrate solution. This was dry spun at a speed of 640 m/min with a speed ratio for the Godet roll to winding machine at 1.30 to obtain 530 decitex multiple filament yarn. The yarn mechanical properties of the obtained yarn are shown in FIG. 1. Properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 5

The same procedures and ingredients as Example 4 were used, but the produced fiber conditions were adjusted to a spinning speed of 558 m/min, with a speed ratio for the Godet roll to winding machine at 1.25 to obtain a 600 decitex multiple filament yarn. The yarn mechanical properties of the obtained yarn are shown in FIG. 1. Properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 6

The same procedures and ingredients as Example 4 were used, but the produced fiber conditions were adjusted to a spinning speed of 488 m/min, with a speed ratio for the Godet roll to winding machine at 1.25 to obtain a 670 decitex multiple filament yarn. The yarn mechanical properties of the obtained yarn are shown in FIG. 1. Resultant properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 7

A prepolymer was obtained by reacting polytetramethylene ether glycol (PTMEG) with a molecular weight of 1402 g/mole with 4,4′-diphenylmethane diisocyanate (MDI) at a 3.50:1.00 (weight by weight), respectively, in a continuous polymerization reactor under neat conditions at 75° C. for 4 h at a specified reaction rate. The residual isocyanate group after the reaction was 3.13 weight %. The capping ratio (mole ratio of isocyanate to glycol) was 1.60. 83.33 grams of the obtained prepolymer were dissolved in 132.70 grams of DMAc at 60° C., and the chain extender solution in which 1.59 grams of ethylenediamine and 0.25 grams of diethylamine and 56.88 grams DMAc was added while stirring vigorously at 80° C. to obtain a viscosity adjusted polymer solution of 31 weight % concentration.

The polymer was blended at 96 parts by weight of the polyurethane polymer solids with 4 parts by weight of the additive solids to make the spinning concentrate solution. This was dry spun at a speed of 549 m/min with a speed ratio for the Godet roll to winding machine at 1.25 to obtain 600 decitex multiple filament yarn. The yarn mechanical properties of the obtained yarn are shown in FIG. 1. Resultant properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 8

A prepolymer was obtained by reacting polytetramethylene ether glycol (PTMEG) with a molecular weight of 1432 g/mole with 4,4′-diphenylmethane diisocyanate (MDI) at a 3.66:1.00 (weight by weight), respectively, in a continuous polymerization reactor under neat conditions at 80° C. for 3 h at a specified reaction rate. The residual isocyanate group after the reaction was 2.79 weight %. The capping ratio (mole ratio of isocyanate to glycol) was 1.53. 130.62 grams of the obtained prepolymer were dissolved in 180.69 grams of DMAc at 60° C., and the chain extender solution in which 2.31 grams of ethylenediamine and 0.32 grams of diethylamine and 66.87 grams DMAc was added while stirring vigorously at 80° C. to obtain a viscosity adjusted polymer solution of 35 weight % concentration.

The polymer was blended at 94 parts by weight of the polyurethane polymer solids with 6 parts by weight of the additive solids to make the spinning concentrate solution. This was dry spun at a speed of 689 m/min with a speed ratio for the Godet roll to winding machine at 1.25 to obtain 530 decitex multiple filament yarn. The yarn mechanical properties of the obtained yarn are shown in FIG. 1. Resultant properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 9

The same procedures and ingredients as Example 8 were used, but the produced fiber conditions were adjusted to a spinning speed of 619 m/min, with a speed ratio for the Godet roll to winding machine at 1.25 to obtain a 600 decitex multiple filament yarn. The yarn mechanical properties of the obtained yarn are shown in FIG. 1. Resultant properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 10

The same procedures and ingredients as Example 8 were used, but the produced fiber conditions were adjusted to a spinning speed of 549 m/min, with a speed ratio for the Godet roll to winding machine at 1.25 to obtain a 670 decitex multiple filament yarn. The yarn mechanical properties of the obtained yarn are shown in FIG. 1. Resultant properties of the laminate are depicted in FIG. 2 (0-150% 4-end laminate cyclic tensile testing, at 3.8×draft levels) and in FIG. 3 (pre-stretched laminate retractive force), along with comparative commercial examples.

### Example 11

A prepolymer was obtained by reacting polytetramethylene ether glycol (PTMEG) with a molecular weight of 650 g/mole with 4,4′-diphenylmethane diisocyanate (MDI) at a 2.00:1.00 (weight by weight), respectively, in a 2-L Pyrex® glass container with a continuous overhead stirring, a heater and a thermocouple temperature measurement under neat conditions at 90° C. for 2 h. The residual isocyanate group after the reaction was 2.60%. The capping ratio (mole ratio of isocyanate to glycol) was 1.30. 375.33 grams of the obtained prepolymer were dissolved in 669.81 grams of DMAc at 50° C., and the chain extender solution in which 114.20 grams of 2.0 milliequivalent per gram of ethylene diamine solution and 6.129 grams of 2.0 milliequivalent per gram of diethylamine solution were added while stirring vigorously to obtain a polymer solution.

The resulting polymer solution was mixed with additives and spun to obtain multiple filament yarn. The yarn mechanical properties of the obtained yarn are shown in FIG. 1.

### Example 12

A prepolymer was obtained by reacting polytetramethylene ether glycol (PTMEG) with a molecular weight of 650 g/mole with 4,4′-diphenylmethane diisocyanate (MDI) at a 1.92:1.00 (weight by weight), respectively, in a 2-L Pyrex® glass container with a continuous overhead stirring, a heater and a thermocouple temperature measurement under neat conditions at 90° C. for 2 h. The residual isocyanate group after the reaction was 2.82%. The capping ratio (mole ratio of isocyanate to glycol) was 1.35. 380.22 grams of the obtained prepolymer was dissolved in 697.78 grams of DMAc at 50° C., and the chain extender solution in which 133.78 grams of 2.0 milliequivalent per gram of ethylene diamine and 2-methyl-1,5-pentanediamine solution with a mole ratio of 9:1 and 4.87 grams of 2.0 milliequivalent per gram of diethylamine solution was added while stirring vigorously to obtain a polymer solution.

The polymer was mixed with additives and spun to obtain multiple filament yarn. The yarn mechanical properties of the obtained yarn are shown in FIG. 1.

### Example 13

A prepolymer was obtained by reacting polytetramethylene ether glycol (PTMEG) with a molecular weight of 650 g/mole with 4,4′-diphenylmethane diisocyanate (MDI) at a 1.86:1.00 (weight by weight), respectively, in a 2-L Pyrex® glass container with a continuous overhead stirring, a heater and a thermocouple temperature measurement under neat conditions at 80° C. for 3 h. The residual isocyanate group after the reaction was 3.36%. The capping ratio (mole ratio of isocyanate to glycol) was 1.40. 600.21 grams of the obtained prepolymer were dissolved in 1294.78 grams of DMAc at 50° C., and the chain extender solution in which 149.04 grams of 10 wt % ethylene diamine/DMAc solution and 8.78 grams of 10 wt % diethyl amine/DMAc solution was added while stirring vigorously to obtain a 30 wt % polymer solution. The end group concentration derived by the diamine compound was 19.5 meq/kg.

The resulting polymer solution was mixed with additives and spun to obtain multiple filament yarn. The yarn mechanical properties of the obtained yarn are shown in FIG. 1.

### Example 14

A prepolymer was obtained by reacting polytetramethylene ether glycol (PTMEG) with a molecular weight of 1000 g/mole with 4,4′-diphenylmethane diisocyanate (MDI) at a 2.76:1.00 (weight by weight), respectively, in a 2-L Pyrex® glass container with a continuous overhead stirring, a heater and a thermocouple temperature measurement under neat conditions at 90° C. for 2 h. The residual isocyanate group after the reaction was 2.775%. The capping ratio (mole ratio of isocyanate to glycol) was 1.45. 340.73 grams of the obtained prepolymer were dissolved in 632.51 grams of DMAc at 50° C., and the chain extender solution in which 110.82 grams of 2.0 milliequivalent per gram of ethylene diamine and 2-methyl-1,5-pentanediamine solution with a mole ratio of 9:1 and 4.35 grams of 2.0 milliequivalent per gram of diethylamine solution was added while stirring vigorously to obtain a polymer solution.

The polymer was mixed with additives and spun to obtain multiple filament yarn. The yarn mechanical properties of the obtained yarn are shown in FIG. 1.

### Example 15

A prepolymer was obtained by reacting polytetramethylene ether glycol (PTMEG) with a molecular weight of 1000 g/mole with 4,4′-diphenylmethane diisocyanate (MDI) at a 2.66:1.00 (weight by weight), respectively, in a 2-L Pyrex® glass container with a continuous overhead stirring, a heater and a thermocouple temperature measurement under neat conditions at 90° C. for 2 h. The residual isocyanate group after the reaction was 3.06%. The capping ratio (mole ratio of isocyanate to glycol) was 1.50. 343.88 grams of the obtained prepolymer were dissolved in 628.91 grams of DMAc at 50° C., and the chain extender solution in which 123.35 grams of 2.0 milliequivalent per gram of ethylene diamine and 2-methyl-1,5-pentanediamine solution with a mole ratio of 9:1 and 4.75 grams of 2.0 milliequivalent per gram of diethylamine solution was added while stirring vigorously to obtain a polymer solution.

The polymer was mixed with additives and spun to obtain multiple filament yarn. The yarn mechanical properties of the obtained yarn are shown in FIG. 1.

### Example 16

A prepolymer was obtained by reacting polytetramethylene ether glycol (PTMEG) with a molecular weight of 1200 g/mole (prepared by blending 62.5 parts by weight of PTMEG with 1000 g/mole molecular weight and 37.5 parts by weight of PTMEG with a molecular weight of 1800 g/mole) with 4,4′-diphenylmethane diisocyanate (MDI) at a 2.91:1.00 (weight by weight), respectively, in a 2-L Pyrex® glass container with a continuous overhead stirring, a heater and a thermocouple temperature measurement under neat conditions at 80° C. for 3 h. The residual isocyanate group after the reaction was 3.38%. The capping ratio (mole ratio of isocyanate to glycol) was 1.65. 535 grams of the obtained prepolymer were dissolved in 1152.04 grams of DMAc at 50° C., and the chain extender solution in which 133.92 grams of 10 wt % ethylene diamine/DMAc solution and 10.52 grams of 10 wt % diethyl amine/DMAc solution was added while stirring vigorously to obtain a 30 wt % polymer solution. The end group concentration derived by the diamine compound was 26 meq/kg.

The resulting polymer solution was mixed with additives and spun to obtain multiple filament yarn. The yarn mechanical properties of the obtained yarn are shown in FIG. 1.

### Example 17

A prepolymer was obtained by reacting polytetramethylene ether glycol (PTMEG) with a molecular weight of 1400 g/mole with 4,4′-diphenylmethane diisocyanate (MDI) at a 3.29:1.00 (weight by weight), respectively, in a 2-L Pyrex® glass container with a continuous overhead stirring, a heater and a thermocouple temperature measurement under neat conditions at 80° C. for 3 h. The residual isocyanate group after the reaction was 3.22 weight %. The capping ratio (mole ratio of isocyanate to glycol) was 1.70. 520 grams of the obtained prepolymer were dissolved in 1122.66 grams of DMAc at 50° C., and the chain extender solution in which 123.86 grams of 10 wt % ethylene diamine/DMAc solution and 12.16 grams of 10 wt % diethyl amine/DMAc solution was added while stirring vigorously to obtain a 30 wt % polymer solution. The end group concentration derived by the diamine compound was 31 meq/kg.

The resulting polymer solution was mixed with additives and spun to obtain multiple filament yarn. The yarn mechanical properties of the obtained yarn are shown in FIG. 1.

### Example 18

A prepolymer was obtained by reacting polytetramethylene ether glycol (PTMEG) with a molecular weight of 1368 g/mole with 4,4′-diphenylmethane diisocyanate (MDI) at a 3.54:1.00 (weight by weight), respectively, in a continuous polymerization reactor under neat conditions at 80° C. for 3 h at a specified reaction rate. The residual isocyanate group after the reaction was 2.72 weight %. The capping ratio (mole ratio of isocyanate to glycol) was 1.543. 105.83 grams of the obtained prepolymer were dissolved in 146.40 grams of DMAc at 60° C., and the chain extender solution in which 1.88 grams of ethylenediamine and 0.26 grams of diethylamine and 54.16 grams of DMAc was added while metered in reactor at 80° C. to obtain a viscosity adjusted polymer solution of 35 weight % concentration. It is to be understood that the units reported in this example represent grams per minute in the continuous polymerization process.

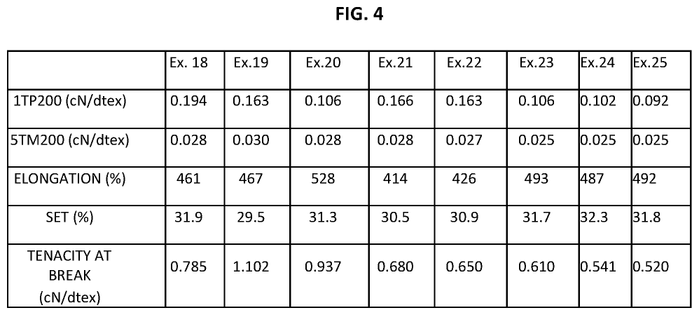

The polymer was blended at 93.25 parts by weight of the polyurethane polymer solids with 6.75 parts by weight of the additive solids to make the spinning concentrate solution. This was dry spun at a speed of 914 m/min with a speed ratio for the Godet roll to winding machine at 1.18 to obtain 33 decitex multiple filament yarn. The yarn mechanical properties of the obtained yarn are shown in FIG. 4.

### Example 19

The same procedures and ingredients as Example 18 were used, but the produced fiber conditions were adjusted to a spinning speed of 869 m/min, with a speed ratio for the Godet roll to winding machine at 1.15 to obtain a 44 decitex multiple filament yarn. The yarn mechanical properties of the obtained yarn are shown in FIG. 4.

### Example 20

The same procedures and ingredients as Example 18 were used, but the produced fiber conditions were adjusted to a spinning speed of 610 m/min, with a speed ratio for the Godet roll to winding machine at 1.18 to obtain a 78 decitex multiple filament yarn. The yarn mechanical properties of the obtained yarn are shown in FIG. 4.

### Example 21

A prepolymer was obtained by reacting polytetramethylene ether glycol (PTMEG) with a molecular weight of 1409 g/mole with 4,4′-diphenylmethane diisocyanate (MDI) at a 3.62:1.00 (weight by weight), respectively, in a continuous polymerization reactor under neat conditions at 80° C. for 3 h at a specified reaction rate. The residual isocyanate group after the reaction was 2.77 weight %. The capping ratio (mole ratio of isocyanate to glycol) was 1.556. 134.28 grams of the obtained prepolymer were dissolved in 185.76 grams of DMAc at 60° C., and the chain extender solution in which 2.38 grams of ethylenediamine and 0.33 grams of diethylamine and 68.73 grams of DMAc was added while metered in reactor at 80° C. to obtain a viscosity adjusted polymer solution of 35 weight % concentration. It is to be understood that the units reported in this example represent grams per minute in the continuous polymerization process.

The polymer was blended at 93.25 parts by weight of the polyurethane polymer solids with 6.75 parts by weight of the additive solids to make the spinning concentrate solution. This was dry spun at a speed of 975 m/min with a speed ratio for the Godet roll to winding machine at 1.28 to obtain 350 decitex multiple filament yarn. The yarn mechanical properties of the obtained yarn are shown in FIG. 4.

### Example 22

The same procedures and ingredients as Example 21 were used, but the produced fiber conditions were adjusted to a spinning speed of 869 m/min, with a speed ratio for the Godet roll to winding machine at 1.25 to obtain a 420 decitex multiple filament yarn. The yarn mechanical properties of the obtained yarn are shown in FIG. 4.

### Example 23

The same procedures and ingredients as Example 21 were used, but the produced fiber conditions were adjusted to a spinning speed of 549 m/min, with a speed ratio for the Godet roll to winding machine at 1.25 to obtain a 670 decitex multiple filament yarn. The yarn mechanical properties of the obtained yarn are shown in FIG. 4.

### Example 24

The same procedures and ingredients as Example 21 were used, but the produced fiber conditions were adjusted to a spinning speed of 457 m/min, with a speed ratio for the Godet roll to winding machine at 1.25 to obtain an 820 decitex multiple filament yarn. The yarn mechanical properties of the obtained yarn are shown in FIG. 4.

### Example 25

The same procedures and ingredients as Example 21 were used, but the produced fiber conditions were adjusted to a spinning speed of 381 m/min, with a speed ratio for the Godet roll to winding machine at 1.25 to obtain a 950 decitex multiple filament yarn. The yarn mechanical properties of the obtained yarn are shown in FIG. 4.

1: An elasticized nonwoven laminate comprising a high recovery power polyurethane elastic fiber and a nonwoven laminate.

2: The elasticized nonwoven laminate of claim 1 wherein the high recovery power polyurethane elastic fiber is adhered within or juxtaposed with the nonwoven laminate.

3: The elasticized nonwoven laminate of claim 1 wherein the high recovery power polyurethane elastic fiber exhibits a normalized recovery force, expressed as the recovery power at 200% elongation of the 5^{th} unload cycle, of at least 0.023 centinewtons per decitex.

4: The elasticized nonwoven laminate of claim 1 wherein the high recovery power polyurethane elastic fiber has a decitex of 30-1500.

5: The elasticized nonwoven laminate of claim 1 wherein the high recovery power polyurethane elastic fiber has a decitex of 33-1100.

6: The elasticized nonwoven laminate of claim 1 wherein the high recovery power polyurethane elastic fiber comprises a polyol, an organic diisocyanate compound, and a diamine compound.

7: The elasticized nonwoven laminate of claim 6 wherein the polyol has a minimum number average molecular weight of 450 and a maximum of 1800.

8: The elasticized nonwoven laminate of claim 6 wherein the polyol has a minimum number average molecular weight of 450 and a maximum of 1600.

9: The elasticized nonwoven laminate of claim 1 wherein the high recovery power polyurethane elastic fiber is adhered within or juxtaposed with the nonwoven laminate via a hot-melt adhesive.

10: The elasticized nonwoven laminate of claim 1 wherein the high recovery power polyurethane elastic fiber is entrapped within or juxtaposed with the nonwoven laminate via an ultrasonic or thermo-mechanical method.

11: An article of manufacture, at least a portion of which comprises the elasticized nonwoven laminate of claim 1.

12: The article of manufacture of claim 11 which is a disposable hygiene product, disposable diaper, training pant or adult incontinence device or product; a catamenial device, garments or product; a bandage, wound dressing, surgical drape, surgical gown, surgical or other hygienic protective mask, hygienic gloves, head covering, head band, ostomy bag, bed pad or bed sheet.

13: The article of manufacture of claim 12 which is a disposable diaper or training pant.

14: The article of manufacture of claim 13 wherein the elasticized nonwoven laminate is positioned at a front panel, back panel, side panel, leg cuff, leg hole, belly band and/or waist band of the diaper or training pant.

15: A method for producing the elasticized nonwoven laminate of claim 1, said method comprising adhering a high recovery power polyurethane elastic fiber within or juxtaposed with the nonwoven laminate.

16: The method of claim 15 wherein the high recovery power polyurethane elastic fiber exhibits a normalized recovery force, expressed as the recovery power at 200% elongation of the 5^{th} unload cycle, of at least 0.023 centinewtons per decitex.

17: The method of claim 15 wherein the high recovery power polyurethane elastic fiber has a decitex of 30-1500.

18: The method of claim 15 wherein the high recovery power polyurethane elastic fiber has a decitex of 33-1100.

19: The method of claim 15 wherein the high recovery power polyurethane elastic fiber comprises a polyol, an organic diisocyanate compound, and a diamine compound.

20: The method of claim 19 wherein the polyol has a minimum number average molecular weight of 450 and a maximum of 1800.

21: The method of claim 19 wherein the polyol has a minimum number average molecular weight of 450 and a maximum of 1600.

22: The method of claim 15 wherein the high recovery power polyurethane elastic fiber is adhered within or juxtaposed with the nonwoven laminate via a hot-melt adhesive.

23: The method of claim 15 wherein the high recovery power polyurethane elastic fiber is entrapped within or juxtaposed with the nonwoven laminate via an ultrasonic or thermo-mechanical method.
